(19) United States
(12) Patent Application Publication
(10) Pub. No.: US 2006/0115848 A1
(43) Pub. Date: Jun. 1, 2006

(54) Nucleic acid end-labeling reagents

(76) Inventors: Andrei Blokhin, Fitchburg, WI (US); Paul M. Slattum, Madison, WI (US); Vladimir G. Budker, Middleton, WI (US)

Correspondence Address: MIRUS CORPORATION, 505 SOUTH ROSA RD, MADISON, WI, 53719, US

(21) Appl. No.: 11/290,802

(22) Filed: Nov. 29, 2005

Related U.S. Application Data
(63) Continuation-in-part of application No. 11/176,405, filed on Jul. 6, 2005.
(60) Provisional application No. 60/586,458, filed on Jul. 8, 2004.

(51) Int. Cl. C12Q 1/68; C07K 14/435; C07H 21/04; C07D 403/02; A61K 38/16

(52) U.S. Cl. 435/6; 536/25.32; 534/727; 530/352; 548/528

(57) ABSTRACT

Compounds and methods are provided for covalent end-labeling of polynucleotides. Incorporation of a nucleic acid affinity group improves the efficiency of reaction of aldehyde reactive groups with the nucleic acid leading to more efficient labeling.

### CROSS-REFERENCE TO RELATED APPLICATIONS

This application is a continuation-in-part of US application Ser. No. 11/176,405, filed 6 Jul. 2005, which claims the benefit of U.S. Provisional Application No. 60/586,458, filed Jul. 8, 2004.

### BACKGROUND OF INVENTION

Labeling of nucleic acids is done for a wide variety of molecular and cellular biology applications. A well-documented application is the use of labeled nucleic acids as probes in detecting specific hybridization events (as in Southern, northern, slot and dot blots, in situ hybridization, and microarrays). Such hybridization applications are a staple of basic research, as well as in diagnostic arenas for the analysis of expression, mutation, polymorphism, and identification of genes, chromosomes or organisms. Other applications of labeled nucleic acids include nucleic acid localization studies, DNA or RNA quantitation, and DNase or RNase quantification. In certain situations it may be particularly desirable to label the nucleic acid at an end of the molecule in such a manner that the base is not affected, because employing this strategy minimizes any effect of the labeling on hybridization properties of the labeled nucleic acid (Laayoun 2003, Agrawal 1986).

There are several existing methods in the art for introducing aldehyde groups into nucleic acids. Aldehydes can be introduced at the 3′ end of RNA via oxidation of the 2′,3′ di-ol functionality of the ribose sugar with the periodate ion (Odom 1980, Hileman 1994, Proudnikov 1996, Millar 1965, Kurata 2003). Aldehyde groups have also been introduced into DNA molecules after partial depurination either as a result of alkylation of N-7 of guanine, treatment with acid, or radical-generating complexes (Kelly 2002, Bavykin 2001, Burrows 1998, Pogozelski 1998, Proudnikov 1996). Following introduction of an aldehyde into a nucleic acid, signaling compounds containing aldehyde-reactive groups can be attached to the nucleic acid. Aldehyde reactive groups include, but are not limited to amines, hydrazines, hydrazides, semicarbazides, and thiosemicarbazides. Hydrazides are of particular interest because the reaction product of a hydrazide and an aldehyde does not require a reduction step for stability (Hansske 1974). Aldehyde-reactive analogs of several signaling compounds have been used to modify nucleic acids. These include tetramethyl rhodamine, fluorescein, biotin, pyrene, anthracene, proflavine, and eosin (Hileman 1994, Proudnikov 1996, Reines 1974, Wu 1996, Odom 1980).

Attachment of signaling compounds has also been done in a three step approach wherein a primary amine is introduced onto the nucleic acid by reacting ethylenediamine with the aldehyde, the resulting imine is then stabilized by reduction, and the introduced primary amine is then reacted with an amine reactive analog of a signaling compound (Broker 1978, Proudnikov 1996).

These labeling methods have the problem of requiring several steps, incomplete reaction of the introduced amine with the fluorophore resulting in less efficient labeling, requiring a large excess of the fluorophore because of electrostatic repulsion, and direct incorporation of the fluorophore being limited to cationic or neutral signaling compounds. Many signaling compounds that are desirable for attachment to nucleic acids bear a negative charge (fluorescein, CyDyes, Alexa Fluors) or are neutral (biotin, rhodamine). Nucleic acids are polyanions and therefore bear a large negative charge. Thus, the electrostatic interaction between nucleic acids and negatively charged signaling compounds is unfavorable and results in low efficiency of reaction. Neutral signaling compounds bear no charge and thus have no electrostatic interaction with the nucleic acid favorable or unfavorable.

MicroRNAs (miRNA) are small, endogenous non-coding RNAs that participate in a variety of natural RNA interference (RNAi) phenomena (Lagos-Quintana et al. 2001, Lau et al 2001, Lee et al. 2001, Lim et al. 2003, Bartel et al 2004). MRNAs modulate the expression of other genes through post-transcriptional effects on target mRNA stability and translational efficiency and may play additional roles in gene regulation. Specific miRNAs are expressed in different cell types, stages of development, and disease states, including certain cancers. MiRNAs have been implicated in several mechanisms of gene regulation in plants, animals, and fungi. In humans, miRNA genes map to chromosomal regions associated with cancer (fragile sites, breakpoints, loss of heterozygocity) and changes in miRNA expression correlate with certain types of cancer (Calin et al. 2002, Calin et al. 2004, Takamizawa et al. 2004, Metzler et al. 2004, Michael et al. 2003). Bioinformatics studies predict that there are ˜250 miRNAs encoded in the human genome (Lim et al. 2003). In any given cell type, only a handful of miRNA genes are expressed at detectable levels (Lagos-Quintana et al. 2002, Lagos-Quintana et al. 2003, Sempere et al. 2004, Liu et al. 2004), indicating that regulation of miRNA expression is highly controlled during differentiation and development.

Because different cell types and disease states are characterized by distinct profiles of miRNA expression, determining miRNA expression profiles has the potential to reveal the roles of miRNAs in human development and cancer. There is a need for high quality miRNA expression profiling tools to facilitate miRNA research. To date, the standard method used to characterize miRNA expression is Northern blotting, a method that is too slow for high throughput analysis. Microarrays represent an ideal option for high-throughput analysis of miRNA expression. Microarrays would theoretically allow all of the human miRNA genes to be profiled in a single experiment, allowing rapid analysis of miRNA expression profiles in different tissues, cell types, and disease states, and, in the future, possibly miRNA diagnostics.

Research on miRNA expression patterns requires the development of new analytical methods and tools because the small size of miRNAs (˜22 nucleotides). The short length of mature miRNAs makes them unlikely candidates for standard microarray labeling protocols that rely on enzymatic replication. The small size of miRNAs precludes the use of typical reverse transcriptase primers and there is no conserved sequence (such as a polyA tail) that allows the use of a universal primer. Priming with short random oligos, while possible (Liu et al. 2004), inevitably leads to variable truncation of the labeled anti-sense strands leading to detrimental effects on microarray hybridization. Other approaches use an enzyme (e.g. terminal nucleotidyl transferase/TdT, poly(A) polymerase, or ligase) to extend the 3′ end of the miRNAs with a modified nucleotide or adapter sequence, which can result in variable melting temperatures for the labeled miRNAs. Finally, 5′ end labeling with poly-nucleotide kinase is limited to radioactive labeling. All of these enzymatic labeling methods are prone to enzymatic biases in the efficiency of replication (or addition) and may preferentially label some sequences over others, so that the labeled material no longer accurately reflects the starting sample.

Current commercially available chemical direct-labeling strategies such as LABELIT® reagents and ULS™ cis-platinum reagents target the N7 position on guanine bases (Slattum et al. 2003, Wiegant et al. 1999). The resulting labeled guanines may affect the melting temperature of the labeled molecule especially for short molecules such as miRNAs. Furthermore, chemical labeling with these methods leads to a variable number of modified bases per molecule generating a heterogeneously labeled miRNA population with variable melting temperatures.

### SUMMARY OF THE INVENTION

In a preferred embodiment, we describe improved nucleic acid labeling compounds comprising: detectable labels, aldehyde reactive groups and an affinity group that increases the affinity of the reagent for negatively charged nucleic acid. Many useful nucleic acid labels are negatively charged. Attachment of these negatively charged labels to negatively charged nucleic acid is therefore inefficient. Increasing the affinity of the reagents to nucleic acid, such as by adding positive charge to the reagent, increases the efficiency of labeling nucleic acid. Reaction of the labeling reagent with nucleic acid results in the formation of a covalent bond between the labeling reagent and the nucleic acid. The described labeling compounds are particularly well suited to end-labeling of nucleic acids, including small RNAs.

In a preferred embodiment of the invention the net charge of the labeling reagent is positive. In another embodiment of the reaction the reagent may be neutral or negative providing the addition of the affinity group decreases the negative charge present on the label or increases the affinity of the label for nucleic acids. For example a label bearing a net negative 2 charge could be conjugated to an aldehyde-reactive group together with an affinity group in such a way that the net charge is now negative 1, neutral, or positively charged. Similarly a label bearing a net negative charge could be conjugated to an aldehyde-reactive group and an affinity group such as a minor groove binder, major groove binder, or intercalating group in such a way that the net charge on the labeling reagent is still negative, but an increased affinity for nucleic acid is achieved.

The label of the present invention may be selected from the group comprising: fluorescence-emitting compounds, radioactive compounds, haptens, immunogenic molecules, chemiluminescence-emitting compounds, proteins, and functional groups. Preferred fluorescence-emitting compounds are fluorescent compounds useful for fluorescence microscopy and microarray analyses.

The labeled nucleic acid can be used for several purposes comprising: 1) techniques to detect specific sequences of polynucleic acids that rely upon hybridization or binding affinity of the labeled polynucleotide to target nucleic acid or protein; including dot blots, slot blots, Southern blots, Northern blots, Southwestern blot, FISH (fluorescent in situ hybridization), in situ hybridization of RNA and DNA sequences, and newly developing combinatorial techniques in which the polynucleic acid is on a “chip” or multiwell or multislot device; 2) labeling polynucleotides that are delivered to cells in vitro or in vivo so as to determine their sub-cellular and tissue location; 3) labeling oligonucleotides that are used as primers in amplification techniques such as PCR (polymerase chain reaction); 4) quantitating polynucleotides; 5) quantitating nucleases (including RNases and DNases) by fluorescence polarization or fluorescence dequenching; 6) sequencing polynucleotides; 7) directly detecting mutations; and, 8) covalently attaching reactive groups to polynucleotides.

In a preferred embodiment, we describe cationic hydrazide and diamine labeling reagents that offer highly efficient, straightforward RNA 3′ end labeling methodology that preserves the original RNA sample. The reagents add a single label to an RNA target 3′ terminus, exhibit no sequence bias in labeling, and have a negligible effect on hybridization or melting temperature. Use of these labeling reagents provides for expedient and accurate miRNA profiling and diagnostics. Cationic hydrazide and diamine 3′ end labeling will be applicable to other sample types, such as fragmented RNA samples or depurinated DNA samples, for which there is a need for efficient, sequence-independent, direct chemical labeling for microarray analysis (Cole et al. 2004) as well as other labeling applications.

Further objects, features, and advantages of the invention will be apparent from the following detailed description when taken in conjunction with the accompanying drawings.

### BRIEF DESCRIPTION OF THE DRAWINGS

FIG. 1. Gel illustrating improved labeling of RNA oligonucleotides with cationic CY™3 fluorescent hydrazides. Panel A shows oligonucleotides detected by fluorescence. Panel B shows the same oligonucleotides stained with SYBR® Gold.

FIG. 2. Gar graph illustrating greater nucleic acid labeling efficiency of cationic hydrazides compared to commercially available negatively charged hydrazides.

FIG. 3. Gel illustrating dependence on aldehyde for labeling RNA oligonucleotides with aldehyde reactive labeling reagents and improved labeling with cationic biotin hydrazides (B and L) over neutral biotin hydrazide (N) as detected by streptavidin gel shift.

FIG. 4. Bar graph illustrating efficiency of labeling of RNA oligonucleotides with cationic linear hydrazide reagents and cationic branched hydrazide reagents.

FIG. 5. Bar graph illustrating correlation of labeling density to aldehyde density in the nucleic acid sample.

FIG. 6. Gel photograph illustrating hydrazide labeling reagent labeling of in vitro transcribed RNA. Panel A shows unstained gel detected by label fluorescence. Panel B shows RNA stained with ethidium bromide.

FIG. 7. Microarray image illustrating usefulness of hydrazide labeled RNA oligonucleotides as probes in microarray gene expression analyses.

FIG. 8. Chart illustrating microRNA expression data obtained using hydrazide labeling nucleic acid probes in a microarray hybridization assay.

### DETAILED DESCRIPTION OF THE INVENTION

Described are improved methods and reagents for labeling nucleic acids. The reagents described are capable of covalently attaching labels to nucleic acids that have been modified to contain an aldehyde group or groups. The reagents described exhibit several improvements over existing reagents found in the literature by virtue of extending the method to new labels and improving the efficiency of attaching those labels currently in the art. A compound suitable for use with the present invention minimally consists of an aldehyde-reactive group, a spacer that imparts additional cationic charge or lessens the negative charge of the reagent or increases the affinity of the reagent for nucleic acids in other manners, and a label (components A-aldehyde reactive group, B-affinity group, and D-label, see below). Suitable compounds may optionally contain a spacer group (component S below). The invention is not limited to a single spatial arrangement of A, B, and D. For example, the labeling reagent can be constructed in a linear arrangement with respect to A, i.e. A-B-D or A-D-B. Alternatively, the labeling reagent may be constructed in a branched arrangement with respect to A, i.e. B-A-D.
A-aldehyde-reactive group: A chemical group that forms covalent bond with carboxylic carbon. For purpose of this invention the reaction product must be sufficiently stable to sustain typical hybridization conditions. Aldehyde reactive group include but not limited to primary or secondary amines, hydrazines, hydrazides, semicarbazides, thiosemicarbazides, oxyamine derivatives and any other nucleophile that yield a stable product in reaction with aldehydes. It should be noted that the addition of primary amines and certain other nucleophiles can be stabilized by a reduction step with various reduction reagents such as sodium borohydride or sodium cyanoborohydride.
B—Affinity group: A group that increases affinity of the reagent for nucleic acid or alters the overall charge of the labeling reagent. The affinity group can be attached to the aldehyde-reactive group, to the label or to the linker/spacer. Alternatively, the affinity group may be incorporated into the linker/spacer. The affinity group may bear a net positive charge or be any of the following: minor groove binders, major groove binders, intercalating groups, or peptides, proteins or groups that increase the affinity of the compound for RNA or other nucleic acids. Any group incorporated into the above compound that increases the affinity of the compound for nucleic acids, thereby increasing the efficiency of the labeling reaction is an affinity group.
D—Label: A label is a reporter group (detectable marker) or functional group.
   reporter group—A chemical moiety attached to the compound for purposes of detection.
   The reporter molecule may be fluorescent, such as a rhodamine, fluorescein derivative or a cyanine dye. The reporter molecule may be a hapten, such as digoxin, or a molecule which binds to another molecule such as biotin which binds to avidin and streptavidin or oligosaccharides which bind to lectins. The reporter molecule may be a protein or an enzyme such as alkaline phosphatase. The reporter molecule may also be or contain radioactive atoms such as ³H, ¹⁴C, ³²P, ³³P, ³⁵S, ¹²⁵I, ¹³¹I, ⁹⁹Tc, and other radioactive elements.
   functional group—a group that adds to or alters the physical behavior of a nucleic acid. This group comprises: reactive groups (excluding those that react with aldehyde-reactive groups), charged groups, alkyl groups, polyethyleneglycol, ligands, and peptides. A reactive group is capable of undergoing further chemical reactions. Reactive groups include, but are not limited to: groups mentioned in A to enable crosslinking to other nucleic acids, alcohols, thiols, acyl azides, carbonates, alkylposphates, carboxylic acids, and other reactive groups that do not react with groups mentioned in A.
S—Linker/Spacer: A connection, typically between the aldehyde-reactive group and the label, made up of a combination of covalent, organometallic, dative, or other chemical bonds containing linkages that may include: alkanes, alkenes, esters, ethers, polyethers, polyethyleneglycols, polypropyleneglycols, glycerol, amides, saccharides, polysaccharides, heteroatoms such as oxygen, sulfur, or nitrogen, and molecules that are cleavable under physiologic conditions such as a disulfide bridges or enzyme-sensitive groups. The spacer may alleviate possible molecular interference by separating the reporter molecule from the aldehyde-reactive group or from the nucleic acid after attachment. The spacer may also contain a group that increases the linkage distance between the label or tag and the aldehyde-reactive agent (A). The spacer may also increase the aqueous solubility of the labeling reagent.

In a preferred embodiment of the invention the net charge of the labeling reagent is positive. In another embodiment the reagent may be neutral. For example, a label bearing a net negative charge is conjugated to an aldehyde-reactive group together with an affinity group in such a way that the net charge on the entire reagent is greater than zero.

We have constructed net positively charged labeling reagents from both neutral and negative signaling compounds. Our new reagents demonstrated higher reaction efficiencies when compared to the currently available neutral or negative analogs. The increased efficiency of the reaction is due to an improved electrostatic interaction between the anionic nucleic acid and the aldehyde-reactive signaling compound. The reagents described consist of a signaling compound, a linker with affinity for nucleic acids, and an aldehyde-reactive group. The reagents described need not be assembled in any particular order to be effective.

Definitions:

1. alkyl group—An alkyl group possesses an sp hybridized carbon atom at the point of attachment to a molecule of interest.
2. stable adduct—A product is stable if it can be used as a probe in a typical hybridization reaction and give usable results. The term hybridization refers to the process in which two single-stranded polynucleotides bind non-covalently to form double- or triple-stranded polynucleotide hybrid that can be detected.
4. nucleophile—A reagent which is attracted to centers of positive charge. A nucleophile participates in a chemical reaction by donating electrons to a species known as an electrophile in order to form a chemical bond. Because nucleophiles donate electrons, they are by definition Lewis bases. All molecules or ions with a free pair of electrons can act as nucleophiles, although negative ions (anions) are more potent than neutral reagents.
4. aldehyde-reactive group—A chemical group that is a nucleophile and is capable of undergoing reaction with an aldehyde to form a covalent bond with carbonylic carbon. For purpose of this invention the reaction product must be sufficiently stable to sustain hybridization conditions. Aldehyde reactive group include but not limited to primary or secondary amines, hydrazines, hydrazides, semicarbazides, thiosemicarbazides, oxyamine derivatives and any other nucleophiles that yield a stable product in reaction with aldehydes. It should be noted that the addition of primary amines and certain other nucleophiles can be stabilized by a reduction step with various reduction reagents such as sodium borohydride or sodium cyanoborohydride. 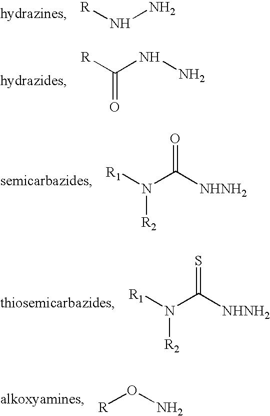
Substituted diamines, hydroxyamines, and mercaptoamines are attractive aldehyde-reactive groups that form stable adducts in reactions with aldehydes (Meyers A I 1992). 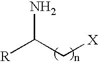
n=1, 2; X═NHR′; OH; SH; R′=H, Alk.
C-nucleophiles (Glitz et al. 1970). 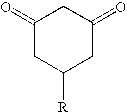
5. alkylation—A chemical reaction that results in the attachment of an alkyl group to the substance of interest.
6. bifunctional—A molecule with two reactive ends. The reactive ends can be identical as in a homobifunctional molecule, or different as in a heterobifucnctional molecule. Bifunctional molecules can be used to cross-link two or more substances together.
7. buffers—Buffers are made from a weak acid or weak base and their salts. Buffer solutions resist changes in pH when additional acid or base is added to the solution.
8. enzyme—Proteins for the specific function of catalyzing chemical reactions.
9. hapten—A small molecule that cannot alone elicit the production of antibodies to itself. However, when covalently attached to a larger molecule it can act as an antigenic determinant, and elicit antibody synthesis. For detection purposes, a hapten is the target of such specific antibodies.
10. hybridization—Highly specific hydrogen bonding system in which guanine and cytosine form a base pair, and adenine and thymine (or uracil) form a base pair.
11. intercalating group—A chemical group characterized by planar aromatic ring structures of appropriate size and geometry capable of inserting themselves between base pairs in double-stranded DNA.
12. label—Labels include reporter or marker molecules or tags such as chemical (organic or inorganic) molecules or groups capable of being detected, and in some cases, quantitated in the laboratory. Reporter molecules may be selected from the group comprising: fluorescence-emitting molecules, immunogenic molecules, haptens (such as digoxin), affinity molecules (such as biotin which binds to avidin and streptavidin), chemiluminescence-emitting molecules, phosphorescent molecules, oligosaccharides which bind to lectins, proteins or enzymes (which produce a signal detectable for example by colorimetry, fluorescence, or luminescence: such as horseradish peroxidase, alkaline phosphatase, β-galactosidase, and glucose-6-phosphate dehydrogenase), and radioactive atoms or molecules. Fluorescence-emitting molecules selected from the list comprising: fluoresceins, rhodamines, cyanine dyes, hemi-cyanine dyes, pyrenes, lucifer yellow, BODIPY®, malachite green, coumarins, dansyl derivatives, mansyl derivatives, dabsyl derivatives, NBD fluoride, stillbenes, anthrocenes, acridines, rosamines, TNS chloride, ATTO-TAG™, LISSAMINE™ derivatives, ALEXA® dyes, eosins, naphthalene derivatives, ethidium bromide derivatives, thiazole orange derivatives, ethenoadenosines, CYDYES™, OREGON GREEN®, CASCADE BLUE®, IR Dyes, Thiazole Orange, BODIPY®-Fl, TAMRA, green fluorescent protein (GFP). Preferably, the fluorescent emitting molecule is detectable in a aqueous solution in a sample containing nucleic acid or protein. Radioactive atoms or molecules may be selected from the list comprising: ³H, ¹⁴C, ³²P, ³³P, ³⁵S, ¹²⁵I, ¹³¹I, and ⁹⁹Tc. Labels also include functional groups which alter the behavior or interactions of the compound or complex to which they are attached. Functional groups may be selected from the list comprising: cell targeting signals, nuclear localization signals, compounds that enhance release of contents from endosomes or other intracellular vesicles (releasing signals), peptides (which include nuclear localization signals, polyArginine, polyHistidine, cell permeable peptides, etc.), hydrophobic or alkyl groups (such as dioleoyl and stearyl alkyl chains), and reactive groups (selected from the list comprising: carboxylic acids, amines, thiols, polyacids, chelators, chelators, peptides, ligands, hydrophobic groups, and PEG). For the purposes of the invention, functional groups does not include aldehyde-reactive groups.
13. labeling—Attachment of a reporter molecule or tag via a chemical bond to a compound of interest such as a nucleic acid or protein.
14. chemical bond—Includes covalent, dative, inorganic, and organometallic bonds.
15. labeling reagent—A compound containing a reporter molecule, label, or tag that can be covalently attached to a nucleic acid or a protein or another aldehyde or active ester-containing group
16. minor groove binding group—A chemical group with an affinity for the minor groove of DNA preferentially over the major groove or phosphodiester backbone because of favorable hydrogen-bonding interactions in addition to cationic charge.
17. major groove binding group—A chemical group with an affinity for the major groove of double stranded DNA preferentially over the minor groove or phosphodiester backbone because of favorable hydrogen-bonding interactions in addition to cationic charge through non-covalent interactions.
18. protein—a molecule made up of 2 or more amino acids. The amino acids may be naturally occurring, recombinant or synthetic.
19. radioactive detectable markers—Radioactive detectable markers are characterized by one or more radioisotopes of phosphorous, iodine, hydrogen, carbon, cobalt, nickel, and the like. Detection of radioactive reporter molecules is typically accomplished by the stimulation of photon emission from crystalline detectors caused by the radiation, or by the fogging of a photographic emulsion.
20. salts—Salts are ionic compounds that dissociate into cations and anions when dissolved in solution. Salts increase the ionic strength of a solution, and consequently decrease interactions between polynucleic acids with other cations.

Activation of the polynucleotide by creation of an aldehyde is performed by chemical methods standard in the art. For example, for an RNA, oxidation of the 2′ and 3′ hydroxyls with periodate generates an aldehyde on the 2′ and 3′ carbons, which can react with a hydrazine derivative, such as hydrazide, to form a hydrazone, resulting in covalent attachment of a label to the 3′ end of the RNA.

After incubation of an RNA sample with sodium periodate to oxidize the 2′ and 3′ hydroxyl groups, unreacted periodate is removed by reaction with sodium sulfite or purification of the oxidized RNA. The oxidized RNA, in a low ionic strength buffer (not containing amines), is then incubated with the hydrazide or amino reagent for several minutes to hours. Labeled RNA is purified from the unreacted labeling reagent by any of a variety of size-appropriate or affinity based molecular biology techniques.

The generation of abasic sites or lesions in DNA or RNA by free radical oxidation, chemical treatment (eg. formic acid, alkylating agents/heat etc.), and incorporation of dUTP during DNA synthesis with subsequent digestion at the uracil sites with uracil-DNA N-glycosylase (UNG) also generates aldehydes for labeling purposes. Examples of conditions for aldehyde formation on a polynucleotide are given in: (Burrows C J et al. 1998, Pogozelski W K et al. 1998, and Bavykin S G et al. 2001).

Hydrazide-based RNA endlabeling reagents can be limited in that the adducts they form with nucleic acids have the potential able to undergo β-elimination reactions, cleaving the label and terminal ribose from the nucleic acid. β-Elimination of the terminal nucleotide, illustrated below, is a reaction that takes place in adducts formed by 2′,3′-oxidized terminal ribose and alkylamines (Rammler D H 1971, Uziel M 1973, Timofeev E et al. 1996).

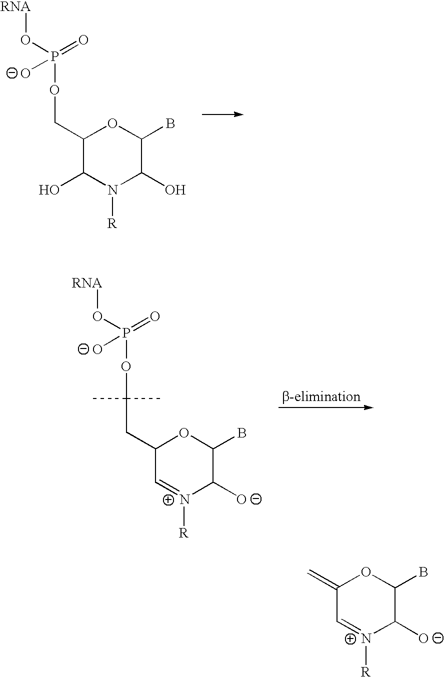

More stable nucleic acid-label adducts (chemical addition products) can be formed with mercaptoamines (I) and diamines (II). These reactive groups interact with carbonyl moieties formed from periodate oxidation of terminal nucleotides to form bicyclic adducts. To attach a label to a nucleic acid, the R group of compounds I, II is chosen to contain the desired label. A preferred linker is designed to work as a spacer between label and RNA and, if necessary, provide positive charge to the molecule. Whether positive charge is incorporated into the spacer depends on the net charge of the label and the net charge, if any, on the reactive group. For instance, diamine reactive groups are themselves positively charged due to protonation at neutral pH, and thus contribute cationic charge to the labeling reagent when combined. A net positive charge on the labeling reagent enhances labeling efficiency through electrostatic interaction with negatively charged nucleic acid.

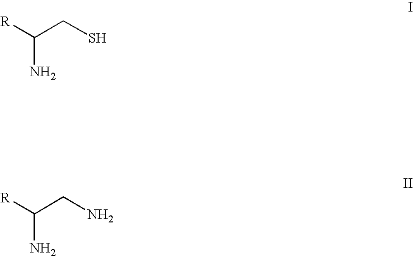

Nucleic acids labeled with reactants I, II are more stable than those formed by alkylamino analogue labeling reagents, because the bicyclic adducts are less likely to participate in α-elimination reactions.

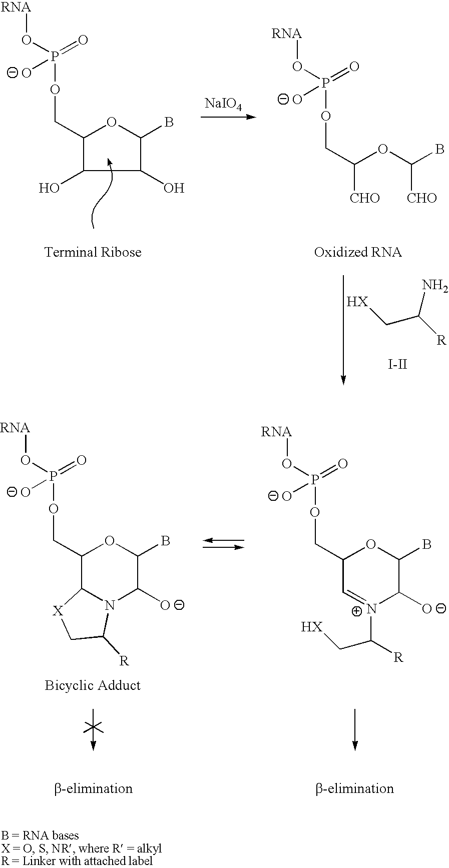

End-labeled RNA or DNA can then be utilized in a number of applications for which labeled nucleic acids are typically used in the art, including use as probes in in situ hybridization reactions, tracking (cellular localization/distribution/function) electrophoretic mobility shift assays, detection of binding proteins, microarrays, Southern blots, Northern blots, dot blots, slot blots, etc.

Any of a large number of nucleic acid sequences may be employed in accord with this invention. Included, for example, are sequences in both RNA and DNA, as are the polynucleotide sequences that characterize various viral, viroid, fungal, parasitic or bacterial infections, genetic disorders or other sequences in target molecules that are desirable to detect. The nucleic acid may be of synthetic, semi-synthetic or natural origin. The described labeling reagents can also be used to label both single stranded and double stranded oligonucleotides, including siRNA and miRNA.

The term polynucleotide, or nucleic acid or polynucleic acid, is a term of art that refers to a polymer containing at least two nucleotides. Nucleotides are the monomeric units of polynucleotide polymers. Polynucleotides with less than 120 monomeric units, or more often less than 50 monomeric units, are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, β-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term polynucleotide includes deoxyribonucleic acid (DNA) and ribonucleic acid (RNA) and combinations of DNA, RNA and other natural and synthetic nucleotides.

DNA may be in form of cDNA, in vitro polymerized DNA, plasmid DNA, parts of a plasmid DNA, genetic material derived from a virus, linear DNA, vectors (P1, PAC, BAC, YAC, artificial chromosomes), expression cassettes, chimeric sequences, recombinant DNA, chromosomal DNA, an oligonucleotide, anti-sense DNA, or derivatives of these groups. RNA may be in the form of oligonucleotide RNA, tRNA (transfer RNA), snRNA (small nuclear RNA), rRNA (ribosomal RNA), mRNA (messenger RNA), in vitro polymerized RNA, recombinant RNA, chimeric sequences, anti-sense RNA, siRNA (small interfering RNA), microRNA (miRNA), ribozymes, or derivatives of these groups. An anti-sense polynucleotide is a polynucleotide that interferes with the function of DNA and/or RNA. Antisense polynucleotides include, but are not limited to: morpholinos, 2′-O-methyl polynucleotides, DNA, RNA and the like. SiRNA comprises a double stranded structure typically containing 15-50 base pairs and preferably 19-25 base pairs and having a nucleotide sequence identical or nearly identical to an expressed target gene or RNA within the cell. Interference may result in suppression of expression. The polynucleotide can be a sequence whose presence or expression in a cell alters the expression or function of cellular genes or RNA. In addition, DNA and RNA may be single, double, triple, or quadruple stranded. Double, triple, and quadruple stranded polynucleotide may contain both RNA and DNA or other combinations of natural and/or synthetic nucleic acids.

A delivered polynucleotide can stay within the cytoplasm or nucleus apart from the endogenous genetic material. Alternatively, DNA can recombine with (become a part of) the endogenous genetic material. Recombination can cause DNA to be inserted into chromosomal DNA by either homologous or non-homologous recombination.

A polynucleotide can be delivered to a cell to express an exogenous nucleotide sequence, to inhibit, eliminate, augment, or alter expression of an endogenous nucleotide sequence, or to affect a specific physiological characteristic not naturally associated with the cell. Polynucleotides may contain an expression cassette coded to express a whole or partial protein, or RNA. An expression cassette refers to a natural or recombinantly produced polynucleotide that is capable of expressing a sequence. The term recombinant as used herein refers to a polynucleotide molecule that is comprised of segments of polynucleotide joined together by means of molecular biological techniques. The cassette contains the coding region of the gene of interest along with any other sequences that affect expression of the sequence of interest. An expression cassette typically includes a promoter (allowing transcription initiation), and a transcribed sequence. Optionally, the expression cassette may include, but is not limited to, transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include, but is not limited to, translation termination signals, a polyadenosine sequence, internal ribosome entry sites (IRES), and non-coding sequences.

### EXAMPLES

### Example 1

### Synthesis of Branched Hydrazide Labeling Reagent

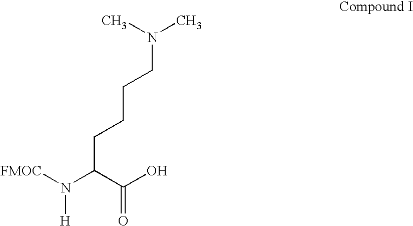

N-α-FMOC-L-Lysine (NovaBiochem, 1.0 g, 2.7 mmol) and formaldehyde (0.520 mL 37% solution, 6.5 mmol) combined in 5.0 mL ethanol. Sodiumcyanoborohydride was added in excess. The reaction was monitored by mass spectrometry and allowed to go to completion. The reaction was acidified with HCl in order to destroy excess cyanoborohydride. The boron salts were removed by filtration and the product was precipitated with ether. The residue was dissolved in methanol and again the precipitated boron salts were removed. The product was precipitated ethyl acetate and vacuum dried to obtain 468 mg product. The structure was verified by mass spectrometry (Sciex API 150 EX) giving a molecular ion (M+1)=397 amu (atomic mass unit).

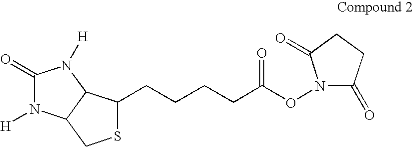

Biotin NHS(N-Hydroxysuccinimide ester) (2) was made by dissolving biotin (2.0 g, 8.1 mmol) in 10 mL N,N-dimethylformamide (DMF). N-hydroxysuccinimide (NHS, 1.1 g, 9.6 mmol) and dicyclohexylcarbodiimide (DCC, 2.0 g, 9.7 mmol) were added and the reaction was stirred at room temperature for two days. The 1,3-Dicyclohexylurea (DHU) was removed by filtration and product was isolated by precipitation with ether yielding 2.1 mg white powder.

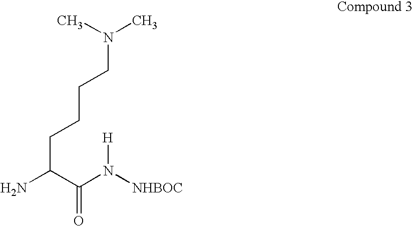

Compound 3 was prepared by initially forming the NHS ester of 1 by dissolving 1 (180 mg, 0.416 mmol) in DMF (0.40 mL) and reacting with NHS (50.0 mg, 0.434 mmol) and DCC (89.4 mg, 0.434 mmol). After removal of the DHU, t-butyl carbazate (Acros, 54.0 mg, 0.416 mmol) was added. The reaction was complete within one hour. The reaction mixture was poured into water. The aqueous solution was washed with ethyl acetate. The aqueous layer was then adjusted to pH 12 and extracted 3 times with ethyl acetate. The combined organic layers were dried over MgSO₄. The solvent was removed in vacuum yielding 40 mg. The FMOC group was removed from the purified product by dissolving in 10% piperidine in DMF. The deprotected product was precipitated with ether yielding 22.5 mg and used without further purification. The structure was verified by mass spectrometry (Sciex API 150 EX) giving a molecular ion (M+1)=289.

### A Branched Biotin Labeling Reagent

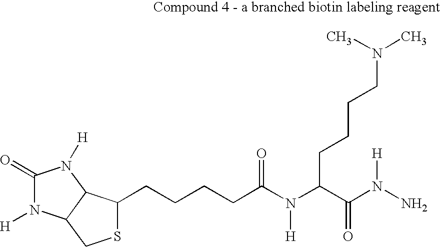

Compound 4 was prepared by combining 3 (22.5 mg, 0.078 mmol) and 2 (26.6 mg, 0.078 mmol) in 0.5 mL DMF using 0.0136 mL diisopropylethylamine (DIPEA) as a proton scavenger. The reaction was complete at the end of one hour. The reaction mixture was purified via HPLC using a C-18 Aquasil column (250×22 mm), mobile phase: acetonitrile (ACN) (0.1% trifluoroacetic acid (TFA)/0.1% TFA, flow=15 mL/min. The product containing fractions were combined and evaporated to dryness. The BOC group was removed by dissolving in TFA and the column purification was then repeated. Removal of mobile phase yielded 3.4 mg pure product. The structure was verified by mass spectrometry (Sciex API 150 EX) giving a molecular ion (M+1)=415 amu.

### Example 2

### Synthesis of a Linear CY3™ Hydrazide Labeling Reagent

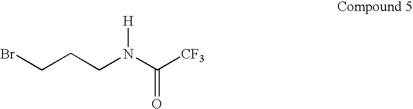

Compound 5 was prepared by adding TFA anhydride (Aldrich Chemical Co., 5.0 mL, 35.4 mmol) dropwise into a stirring solution of 3-bromopropylamine hydrobromide (Aldrich Chemical Co., 7.5 g, 34.0 mmol) and diisopropylethylamine (DIPEA, 12.0 mL, 68.9 mmol) in 200 ml dichloromethane. The reaction was stirred overnight at room temperature. The reaction mixture was washed twice with saturated sodium bicarbonate, twice with water, and once with brine. The product crystallized after removal of solvent. The yield was 8.0 mg.

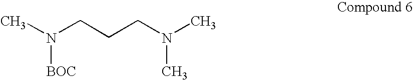

Compound 6 was prepared from N,N,N′-trimethylpropanediamine (Aldrich Chemical Co., 2.5 g, 21.6 mmol) and Boc anhydride (Aldrich Chemical Co., 5.17 g, 23.8 mmol) in ice cold THF (8.0 mL) with diisopropylethylamine (3.8 mL, 21.6 mmol). The reaction was stirred overnight at room temperature. Following solvent removal the reaction residue was taken up in ethyl acetate and washed three times with saturated sodium carbonate and once with brine. Solvent removal and vacuum drying yielded 3.7 g product. The structure was verified by mass spectrometry (Sciex API 150 EX) giving a molecular ion (M+1)=217 amu.

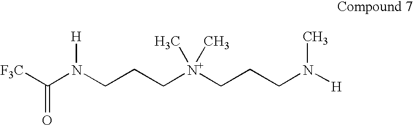

Compound 7 was prepared by alkylating 6 (0.920, 4.2 mmol) with 5 (1.0 g, 4.3 mmol) in DMF (4.0 mL) at 75° C. overnight. The product was isolated by precipitation with ether. The BOC group was removed with TFA, followed by vacuum drying. Structure was verified by mass spectrometry (Sciex API 150 EX) giving a molecular ion (M+)=370 amu.

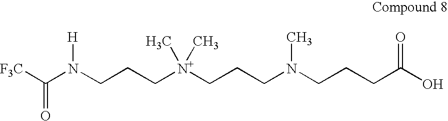

Compound 8 was prepared by combining 7 (192 mg, 0.415 mmol) and succinic semialdehyde (Aldrich Chemical Company, 282 μL, 42.3 mg, 0.415 mmol) in 0.5 mL methanol. After 30 minutes excess sodium cyanoborohydride was added. The reaction was complete within 2 hours. HCl was added to destroy excess borohydride. The boron salts were removed by filtration and the product was isolated by precipitation with ethyl ether. Structure was verified by mass spectrometry (Sciex API 150 EX) giving a molecular ion (M+)=356 amu.

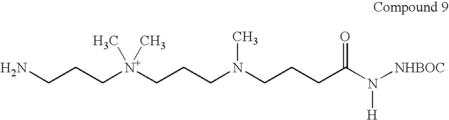

Compound 9 was prepared by initially forming the NHS ester of 8 by dissolving 8 (18.0 mg, 0.082 mmol) in DMF (0.2 mL) and reacting with NHS (9.4 mg, 0.74 mmol) and DCC (16.9 mg, 0.082 mmol). The reaction was complete within 2 hours. The DHU was removed by filtration, and the product was precipitated with ether. The residue was dissolved in DMF (0.2 mL) and t-butyl carbazate (Acros, 6.5 mg, 0.049 mmol) was added. The reaction was complete within one hour. The trifluoroacetamide product precipitated with ether. The trifluoroacetamide group was removed in 0.1 M ammonium carbonate at 90° C. overnight. After lyophilization 14.5 mg product was isolated. Structure was verified by mass spectrometry (Sciex API 150 EX) giving a molecular ion (M+)=374.

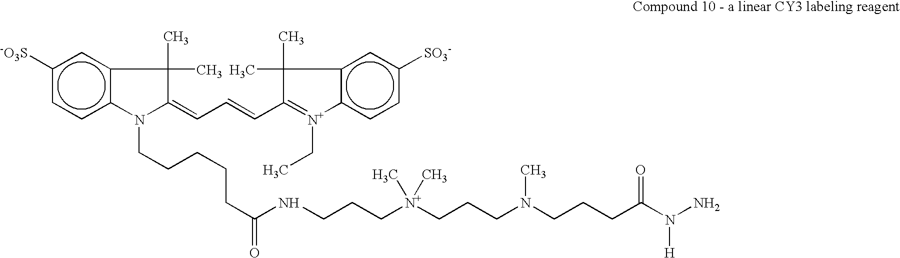

Compound 10 was prepared by combining 9 (14.5 mg, 0.032 mmol), CY®3 NHS ester (Amersham, 30.0 mg, 0.039 mmol) and DIPEA (4.5 μL) in 250 μL DMF. The reaction was complete within one hour. The reaction product was precipitated with ether. The BOC group was removed from the hydrazide with TFA for 15 minutes. The reaction mixture was purified via HPLC using a C-18 Aquasil column (250×22 mm), mobile phase: Methanol/5 mM ammonium formate pH 4.0, flow=15 mL/min. The product containing fractions were combined and evaporated to dryness. Removal of mobile phase yielded 10.6 mg pure product. The structure was verified by mass spectrometry (Sciex API 150 EX) giving a molecular ion (M⁺)=886 amu.

Using similar methods CY5™ and Fluorescein were conjugated to Compound 9.

### Example 3

### An Alternative Linear Fluorescent Hydrazide Polynucleotide Labeling Reagent

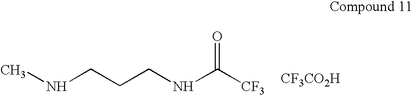

Compound 11 was prepared by acylation of N-methy-1,3-propanediamine (5 g, 56.7 mmol) with ethyltrifluoroacetate (20.14 g, 141.8 mmol) in a mixture of acetonitrile (90 mL) with H₂O (1.15 g, 64 mmol). The reagents were mixed at 0° C., than the reaction mixture was stirred at RT for 1 h, and heated ON at 45° C. The product was concentrated in vacuum to dryness at 40° C. and recrystallized from ethylacetate (EtOAc). Yield 10.3 g, 61%. NMR (Bruker 250, D₂O): 1.98 m (2H), 2.73 s (3H), 3.08, 3.08 m (2H), 3.44 t (2H). MS (Sciex API 150 EX): 185.0 (M+H⁺), 369 (2M+H⁺), 483.2 (2M+H⁺+CF₃CO₂H).

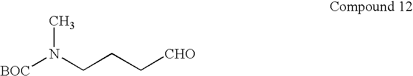

Compound 12. γ-(N-Boc-methylamino)butyraldehyde was prepared from γ-(N-Boc-methylamino)butyric acid according procedure of Xiangshu X et al. 2004.

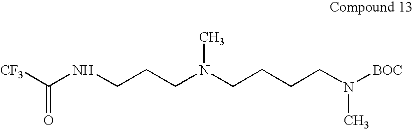

Compound 13 was prepared by stirring a mixture of 11 (2.65 g, 8.89 mmol), 12 (1.75 g, 8.70 mmol), and triethylamine (TEA, 1.86 g, 2.56 mmol) in dichloromethane (30 mL) for 30 min. NaHB(OAc)₃ (2.58 g, 12.17 mmol) was added, stirred for 10 h and cooled to 0° C. A saturated K₂CO₃ aqueous solution (10 mL) was added, the organic phase was separated, and the aqueous was extracted with CHCl₃ (6×5 mL). The organic phases were combined, dried (MgSO₄), filtered, concentrated and dried in vacuum. The product was purified on a column (SiO₂, CHCl₃:methanol=9:1). Yield 2.77 g, 86%. NMR (CDCl₃): 1.45 s (9H), 1.50 m (4H), 1.72 m (2H), 2.22 s (3H), 2.39 m (2H), 2.55 m (2H), 2.82 s (3H), 3.22 m (2H), 3.47 m (2H), 9.68 bs (1H). MS=370 amu (M+H⁺).

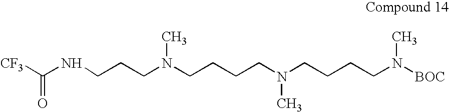

Compound 14 was prepared by initial Boc-deprotection of 13 (500 mg, 1.35 mmol) in a 1:1 mixture of TFA-CH₂Cl₂ (2 mL). The dry product was stirred with 12 (272 mg, 1.35 mmol) and TEA (273 mg, 2.7 mmol) for 30 min in dichloroethane (6 mL), cooled to 5° C. and treated with NaH(OAc)₃. The reaction mixture was stirred at RT for 10 h, and basified with aq. K₂CO₃ at 0° C. to pH=11. The product was dried (MgSO₄), filtered, concentrated and purified on a column (SiO₂, CH₂Cl₃:methanol:NH₄OH=9:1:0.02). Yield 504 mg, 82%. NMR (CDCl₃): 1.45 s (9H), 1.55 m (8H), 1.72 m (2H), 2.10-2.8 m (8H), 2.22 s (3H), 2.82 s (3H), 3.22 m (2H), 3.48 m (2H), 9.39 bs (1H). MS=455.4 amu (M+H⁺).

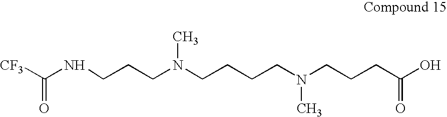

Compound 15 was prepared from 13 (292 mg, 0.789 mmol) following deprotection with TFA (0.5 mL) in CH₂Cl₂ (1 mL) for 40 min. The deprotected amine was dried in vacuum, dissolved in H₂O (0.5 mL), basified with K₂CO₃ to pH=7, and dried vacuum. The residue was dissolved in methanol (4 mL), succinic semialdehyde (aq. solution 15%, 0.5 mL, 0.789 mmol) was added, and the mixture was treated with NaCNBH₃ (1 M solution in THF, 1.1 mL, 1.1 mmol). In 4 h the reaction mixture was concentrated in vacuum and quenched with 1N HCl. MS=378 amu (M+Na⁺). The crude product was used directly in the next step.

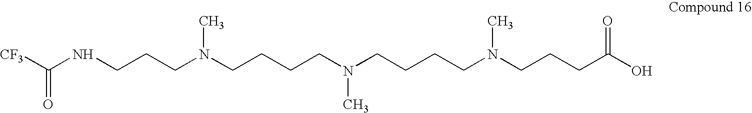

Compound 16 was prepared from 14 (242 mg, 0.532 mmol) following deprotection with TFA (1 mL) in CH₂Cl₂ (1 mL) for 40 min. The deprotected amine was dried in vacuum, dissolved in H₂O (0.5 mL) and basified with K₂CO₃ to neutral pH, and concentrated in vacuum. The residue was dissolved in methanol (4 mL), succinic semialdehyde (aq. solution 15%, 0.335 mL, 0.532 mmol) was added, and the mixture was treated with NaCNBH₃ (1 M solution in THF, 0.745 mL, 0.745 mmol). In 4 h the reaction mixture was concentrated in vacuum and quenched with 1N HCl. MS: 441 amu (M+H⁺). The crude product was used directly in the next step.

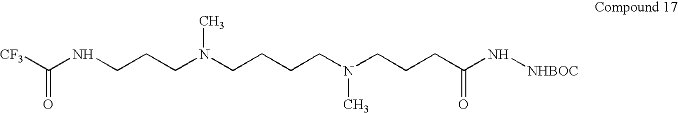

Compound 17 was prepared by from acid 15 (240 mg, 0.67 mmol) via conversion to NHS ester by treatment with DCC (303 mg, 1.47 mmol) and 1,3-dicyclohexyl-carbodiimide (162 mg, 1.47 mmol) in DMF (4 mL) for 10 h. The formation of the NHS derivative was confirmed by MS: 454.5 (M+H⁺). A solution of t-butyl carbazate (133 mg, 1.01 mmol) in DMF (0.5 mL) was added to the reaction mixture, stirred for 2 h, DMF was removed in vacuum, and the product, 17 was purified on column (SiO₂, CH₂Cl₂:methanol:NH₄OH=8:2:0.1-8:2.5:0.1). Yield 251 mg, 80%. NMR (D₂O): 1.47 s (9H), 2.06 m (4H), 2.44 m (2H), 2.89 s (3H), 2.90 s (3H), 3.22 m (8H), 3.44 m (2H). MS: 470 amu (M+H⁺).

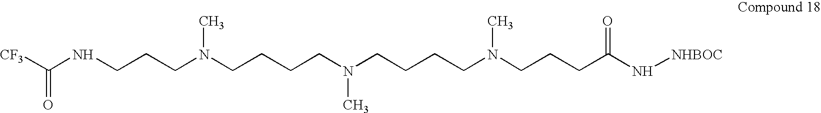

Compound 18 was prepared by from acid 16 (234 mg, 0.532 mmol) via converting it to NHS ester by treatment with DCC (274 mg, 1.33 mmol) and 1,3-dicyclohexylcarbodiimide (123 mg, 1.07 mmol) in DMF (5 mL) for 10 h. The formation of the NHS derivative was confirmed by MS: 538.4 (M+H⁺). A solution of t-butyl carbazate (175 mg, 1.33 mmol) in DMF (0.5 mL) was added to the reaction mixture, stirred for 2 h, DMF was removed in vacuum, and the product, 18, was purified on column (SiO₂, CH₂Cl₂:methanol: NH₄OH=6:4:0.2). Yield 184 mg, 62%. NMR (D₂O): 1.47 s (9H), 1.67 m (8H), 1.92 m (4H), 2.38 m (2H), 2.50 s (3H), 2.58 m (2H), 2.65 s (3H), 2.66 s (3H), 2.7-3.2 m (10H), 3.38 m (2H). MS: 555 amu (M+H⁺).

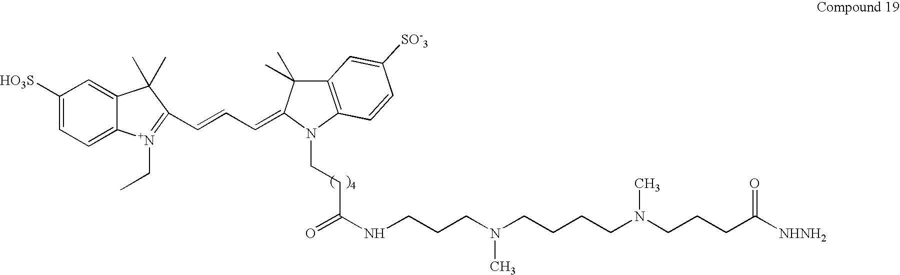

Compound 18 was prepared from 17 that was initially TFA deprotected. A 5% solution of 17 in a 1:1 mixture of conc. aqueous NH₄OH in methanol was refluxed for 5 h, then concentrated and dried in vacuum to free terminal NH₂ group. The deprotected amine (3.5 mg, 0.00937 mmol) was dissolved in DMF (35 μL) and stirred for 1.5 h with a solution of CYβ3-NHS ester (Amersham, 6.81 mg, 0.00937 mmol) and DIPEA (1.21 mg, 0.00937 mmol) in DMF (80 μL). Et₂O (1.5 mL) was added, the precipitate was separated and purified via HPLC using C-18 Aquasil column (250×4.6 mm), mobile phase MeOH (TFA 0.1%)—H₂O (TFA 0.1%), flow 1 mL/min. The formation of the product of coupling was confirmed by MS: 986.6 (M+H⁺). Boc-deprotection by stirring in a mixture of methanol-20% HCl (2:1, 0.2 mL) for 15 h at RT, followed by concentration in vacuum and purification via HPLC (C-18 Aquasil column (250×4.6 mm), mobile phase methanol (TFA 0.1%)/H₂O (TFA 0.1%), flow 1 mL/min) yielded XIX in an amount of 0.8 mg. MS: 886 amu (M+1). NMR (D₂O): 1.22 m (2H), 1.35 t (3H), 1.56 m (2H), 1.72 s (12H), 1.65-1.90 m (6H), 1.85 m (2H), 2.02 m (2H), 2.15 t (2H), 2.42 t, (2H), 2.77 s (3H), 2.85 s (3H), 2.87 m (2H), 2.9-3.3 m (8H), 4.13 m (4H), 6.36 m (2H), 7.37 m (2H), 7.82 m (4H), 7.88 m (2H), 8.52 m (1H).

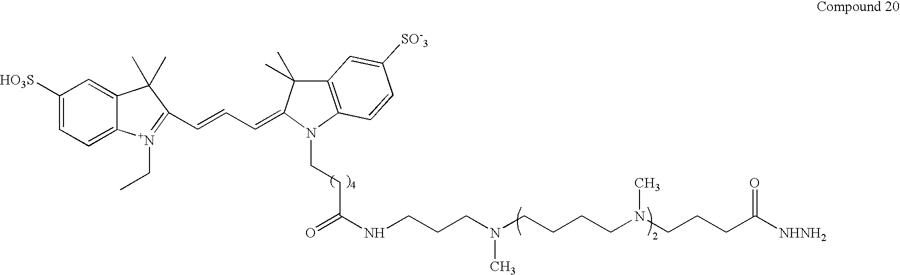

Compound 20 was prepared from 18 using the procedure described for preparation of 19. MS: 971 amu (M+H⁺). NMR (D₂O): 1.29 m (2H), 1.36 t (3H), 1.58 m (2H), 1.72 s (12H), 1.7-1.9 m (12H), 2.05 m (2H), 2.17 t (2H), 2.45 t (2H), 2.79 s (3H), 2.87 s (3H), 2.88 s (3H), 2.85-3.35 m (14H), 4.12 m (4H), 6.37 m (2H), 7.37 m (2H), 7.82 m (2H), 7.89 m (2H), 8.51 m (1H).
A Linear Biotin Hydrazide Polynucleotide Labeling Reagent.

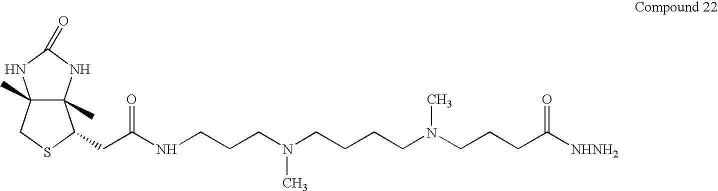

Compound 22 was prepared from 17 that was initially TFA-deprotected. A 5% solution of 17 in a 1:1 mixture of conc. aqueous NH₄OH in methanol was refluxed for 5 h, than concentrated and dried in vacuum to free terminal NH₂ group. The deprotected amine (19.4 mg, 0.052 mmol) was dissolved in DMF (2 mL) and stirred with Biotin-NHS ester (18 mg, 0.053 mmol) and DIEA (20 mg, 0.156 mmol) for 1.5 h. The product was concentrated in vacuum, triturated with hexane and purified via HPLC using C-18 Aquasil column (250×22 mm), mobile phase CAN (0.1% TFA)/0.1% TFA, flow 15 mL/min. Final Boc-deprotection was done by stirring in pure TFA (0.5 mL) for 30 min. TFA was removed in vacuum and the product 22 was purification via HPLC (C-18 Aquasil column (250×22 mm), mobile phase CAN (0.1% TFA)/0.1% TFA, flow 15 mL/min.). Yield 12 mg. Mass spectrum: 500 amu (M+H⁺).

### Example 4

### Synthesis of a Branched CY3™ Hydrazide Labeling Reagent

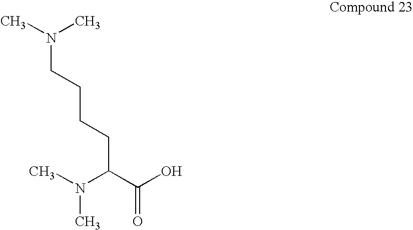

Compound 23 was prepared byalkylation of L-Lysine (1.0 g, 6.8 mmol) with formaldehyde (37% aqueous, 2.2 mL, 27.4 mmol) in 10 mL methanol. Sodiumcyanoborohydride was added in excess. The reaction was stirred at room temperature for 3 hours. The pH of the reaction was then adjusted to approximately 2 with HCl (1 N) to destroy excess borohydride. The precipitated boron salts were removed by filtration and solvent was removed to yield 1.9 g as a clear oil. The structure was confirmed by mass spectrometry giving a signal for the molecular ion (M+1)=203 amu.

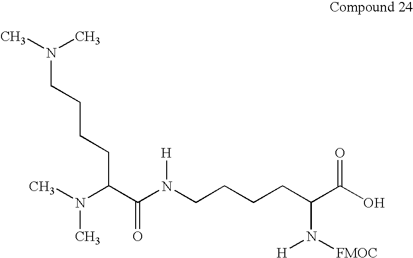

Compound 24 was prepared by initially forming the NHS ester of 23 by dissolving 23 (207 mg, 0.76 mmol) in DMF (4.0 mL) and reacting with NHS (131 mg, 1.14 mmol), DCC (240 mg, 1.16 mmol), and DIPEA (0.132 mL, 0.76 mmol). The reaction was stirred under nitrogen and was complete within 1 hour. The DHU was removed by filtration, and the product was precipitated with ether and vacuum dried to yield 201 mg. The product identity was confirmed by mass spectrometry giving a signal for the molecular ion (M+1) of 300. The NHS ester (201 mg, 0.67 mmol) was combined with N-α-FMOC-L-Lysine (Novabiochem, 200 mg, 0.67 mmol) in DMF (4.0 mL). The reaction was stirred under nitrogen at room temperature and was finished within 2 hours. The reaction mixture was purified via HPLC using a C-18 Aquasil column (250×22 mm), mobile phase: ACN (0.1% TFA)/(0.1% TFA), flow=15 mL/min. The product containing fractions were combined and evaporated to dryness. Removal of mobile phase yielded 210 mg pure product. The structure was verified by mass spectrometry (Sciex API 150 EX) giving a molecular ion (M+1)=553 amu.

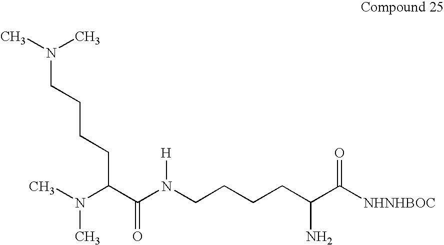

Compound 25 was prepared by initially forming the NHS ester of 24 by dissolving 24 (125 mg, 0.226 mmol) in DMF (2.0 mL) and reacting with NHS (40 mg, 0.35 mmol) and DCC (60 mg, 0.29 mmol). The reaction was stirred at room temperature under nitrogen overnight. The DHU was removed by filtration, and the product was precipitated with ether. The residue was dissolved in DMF (1.0 mL) and t-butyl carbazate (Acros, 26 mg, 0.20 mmol) was added. The reaction was complete within one hour. The product precipitated with ether and was purified via HPLC using a C-18 Aquasil column (250×22 mm), mobile phase: ACN (5 mM trimethylamine/formic acid pH 4.0)/(5 mM trimethylamine/formic acid pH 4.0), flow=15 mL/min. The product containing fractions were combined and evaporated to dryness. Removal of mobile phase yielded 14.0 mg pure product with FMOC group removed. Structure was verified by mass spectrometry (Sciex API 150 EX) giving a molecular ion (M+1)=445 amu.

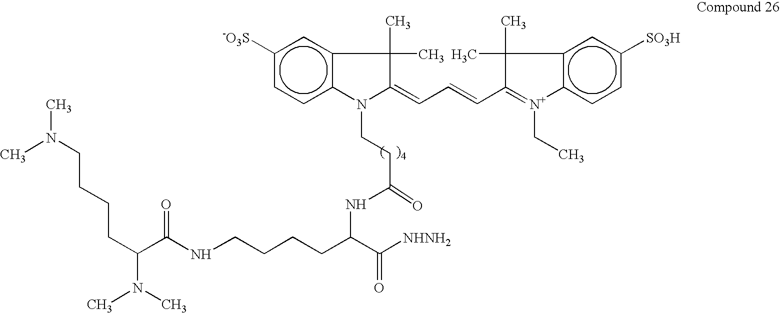

Compound 26 was prepared by combining 25 (7.0 mg, 0.016 mmol), CY®3 NHS ester (Amersham, 12 mg, 0.016 mmol) and DIPEA (10 μL) in 400 μL DMF. The reaction was complete within one hour. The reaction product was precipitated with ether. The BOC group was removed from the hydrazide with TFA for 15 minutes. The reaction mixture was purified via HPLC using a C-18 Aquasil column (250×22 mm), mobile phase: Methanol (0.1% TGA)/0.1% TFA, flow=15 mL/min. The product containing fractions were combined and evaporated to dryness. The structure was verified by mass spectrometry (Sciex API 150 EX) giving a molecular ion (M+1)=957 amu.

A CY®5 analog of compound 26 was prepared in the same manner.

### Example 5

### Improved Hydrazide Labeling of Nucleic Acid

RNAs larger than 200 bases were fragmented in pools (50-100 μg) by adding RNA to 20 μl buffer (200 mM Tris, 100 mM MgCl₂), 2 μl 100 mM DTT, 2 μl 5M NaCl, 8 μl SHORTCUT® RNase III (New England BioLabs) and nuclease-free water up to 200 μl total volume. The reactions were incubated at 37° C. for 35 minutes. RNase was inactivated by incubation at 65° C. for 20 min. The resultant fragmented RNAs were purified using MIRVANA™ miRNA isolation kit (Ambion) using 1 volume Lysis/Binding buffer and 50% ethanol (final concentration). Manufacturer's recommended procedure was followed for binding and elution of the fragmented cRNA.

Freshly prepared 50 mM NaIO₄ (1 μl) and 1 μl 50 mM NaOAc (pH 5.5) were added to RNA (1-5 μg; synthetic oligonucleotides, fragmented RNA, or small RNA sample in 10 μl nuclease-free water). Solutions were incubated for 1 hour at room temperature. Excess NaIO₄ was neutralized with 1 μl freshly prepared Na₂SO₃ for 30 minutes. Labeling reactions were carried out for 1 to 4 hours at room temperature in 10011 with the addition of CY™3, CY™5, or Biotin hydrazide reagents at 8-16 μM final concentration (5-10 fold excess, assuming 1 μg of 21-mer RNAs for the linear hydrazides) or 80 μM final concentration (50 fold excess, assuming 1 μg of 21-mer RNAs for the branched hydrazides). Total reaction volumes were brought to 200 μg with nuclease-free water and samples were precipitated with the addition of 20 μg glycogen, 20 μl 5M NaCl and 500 μl ice cold 100% Ethanol. Pellets were washed and immediately resuspended in 60 μl 2 mM MOPS pH 7.5. Spectrophotometric and gel analysis allowed assessment of labeling efficiency.

### Example 6

### Cationic Hydrazides Exhibit Superior RNA End-Labeling Performance

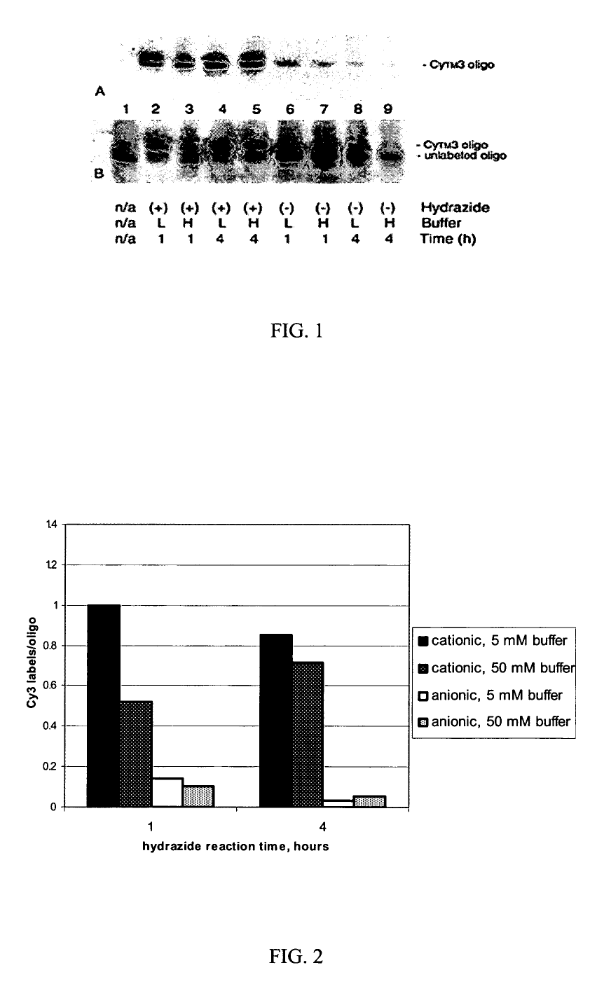

A synthetic 21-mer RNA oligo was labeled, in duplicate, according to the standard labeling reaction conditions described in Example 5 using ˜15 μM of either the linear cationic (+) or commercial anionic (−) CY™3-hydrazide (GE Healthcare) for 1 or 4 hours at room temperature. The end-labeling reactions were either performed in 5 mM (L, low ionic strength) or 50 mM (H, high ionic strength) sodium phosphate buffer, pH 7.5. 50 ng purified RNA (from representative samples of each treatment) were resolved by electrophoresis using a 20% NOVEX®/TBE native acrylamide gel (Invitrogen). An image of the unstained gel indicates the CY™3 signal from labeled oligos (FIG. 1, panel A); an image of the same gel after staining with SYBR® Gold (Invitrogen) indicates both CY™3-labeled and unlabeled oligos (FIG. 1, panel B); lane 1 contains an unlabeled sample. CY™3-labeled oligos exhibit reduced electrophoretic migration and are detected predominantly in the cationic hydrazide samples. The average labeling efficiency (number of CY™3 labels per oligo) (FIG. 2) of each treatment was calculated based on spectrophotometric analysis of the purified samples, indicating the improvement of labeling efficiency with the cationic Cy™3-hydrazide, particularly in low ionic strength conditions.

### Example 7

### Increased Efficiency of Cationic Linear Biotin Hydrazide and Cationic Branched Hydrazide Over Neutral Commercially Available Biotin Hydrazide

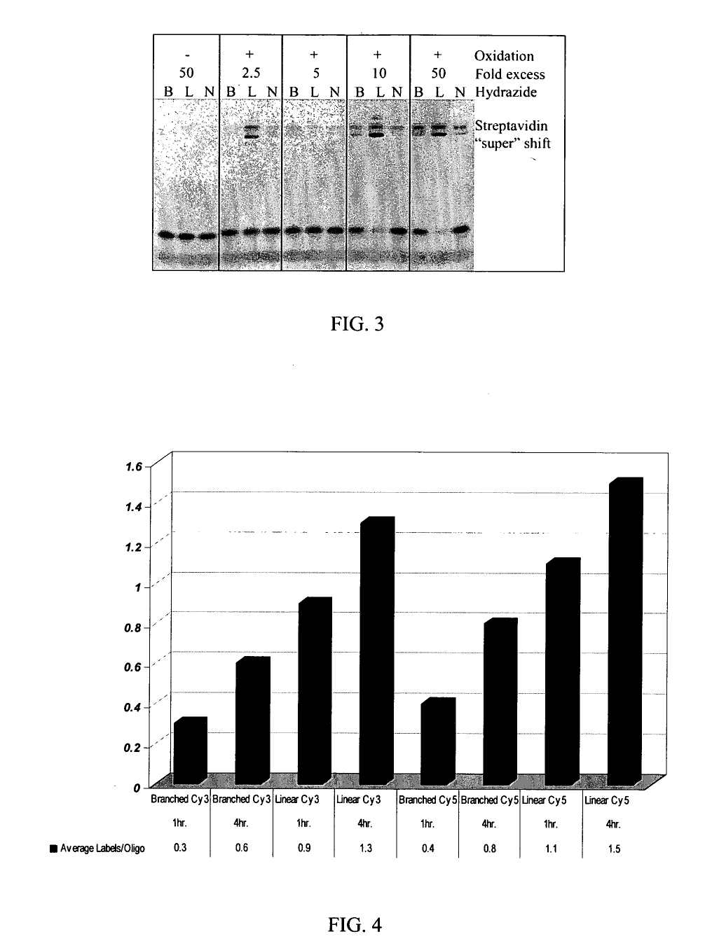

Labeling reactions of a synthetic 21-mer RNA oligo were performed with increasing molar excess of cationic branched (B), cationic linear (L), or neutral biotin hydrazides (N) as described in example 5. Neutral biotin hydrazide was purchased from Sigma, Inc. Samples were labeled with the indicated hydrazides (B, L, or N), precipitated and assayed for biotin incorporation using streptavidin conjugation and a gel shift assay (FIG. 3). 75 ng aliquots of each sample were separate on a 20% acrylamide gel and stained with SYBRE Gold nucleic acid gel stain (Invitrogen). Samples demonstrated biotin labeling incorporation with a “super shift” or reduced electrophoretic migration of streptavidin-bound labeled RNA. Non-oxidized negative control RNA oligos showed no incorporation of biotin hydrazides. Cationic hydrazides show increased efficiency of labeling over the neutral commercially available hydrazide reagent.

### Example 8

### RNA Oligonucleotide Labeling with Cationic Linear Hydrazides and Cationic Branched Hydrazide Reagents

Labeling reactions with cationic linear and branched CY™3 and CY™5 hydrazides on a synthetic 21-mer RNA oligo were performed as described in example 5. Samples were labeled with 10-fold molar excess linear hydrazide or 50-fold molar excess branched hydrazide and incubated for 1 or 4 hours. Spectrophotometric analysis of CY™3 and CY™5 labeling incorporation (FIG. 4) show linear hydrazides resulting in more efficient labeling compared to the branched hydrazides.

### Example 9

### Cationic Hydrazide Labeling of RNA

Total RNA was isolated from HeLa cells, and used to generate cRNA (modified Eberwine antisense RNA amplification). HeLa cRNA was RNAse III fragmented and 5 μg were labeled with CY™3 cationic hydrazide in parallel to 5 μg unfragmented cRNA and a 1 μg synthetic 21mer RNA oligo according to procedures outlined in Example 5. Non-oxidized RNA samples were exposed to cationic hydrazide reagent as negative controls, demarked as (−). Labeling efficiency was assessed using absorbance detection of the CY™3 fluorophore.

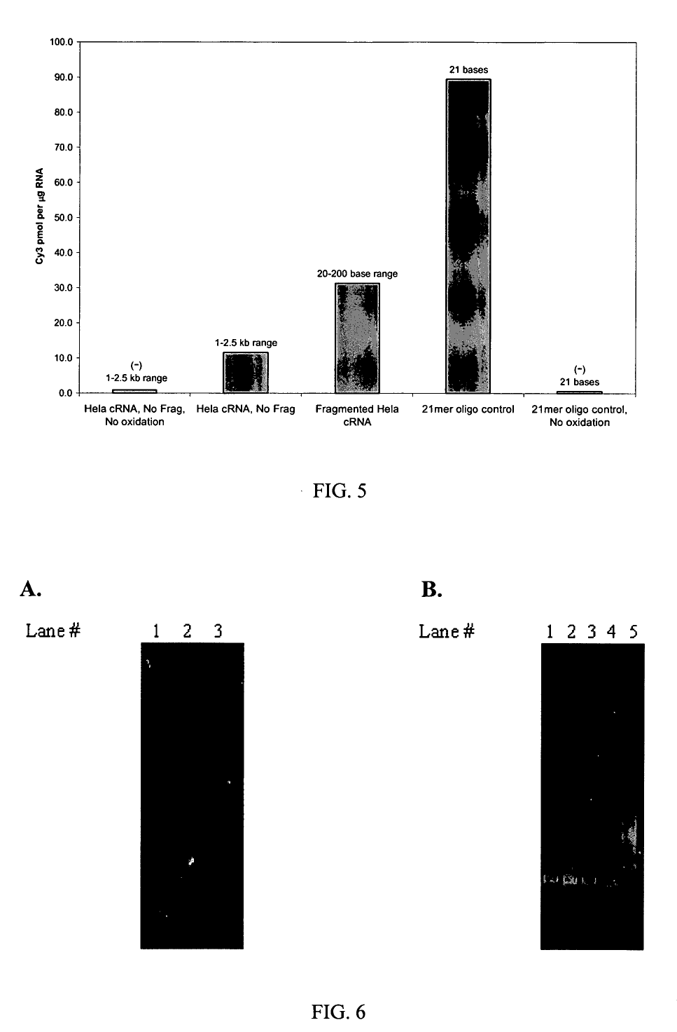

Results, as shown in FIG. 5, demonstrated efficient labeling of oxidized RNA. RNA without aldehydes (i.e. unoxidized) was not labeled. FIG. 5 illustrates incorporated CY™3 per μg RNA (Y axis) for each labeled sample type (X axis). Approximate size range of each sample type is shown above each bar. As expected, increased incorporation is observed in samples containing a higher molar concentration of 3′ ends (aldehydes) per μg sample RNA.

### Example 10

### CY™3 Cationic Hydrazide Labeling of In Vitro Transcribed RNA

Non-oxidized and oxidized in vitro transcribed RNA were exposed to CY™3 cationic hydrazide according to procedures outlined in Example 5, and loaded onto an 0.8% agarose (1×TAE) gel. FIG. 6 illustrates unstained agarose gel loaded with 500 ng RNA per lane (left), and demonstrates no labeling of the non-oxidized control (lane 1), and CY™3 labeling of duplicate oxidized, labeled samples (lanes 2 and 3). Ethidium bromide stained agarose gel loaded with 250 ng RNA per lane (right) shows intact in vitro transcribed RNA in untreated (lane 1), non-oxidized (lane 2), and duplicate CY™3 labeled samples (lanes 3 and 4).

### Example 11

### Hybridization of Cationic Linear CY™3 Labeled cRNA

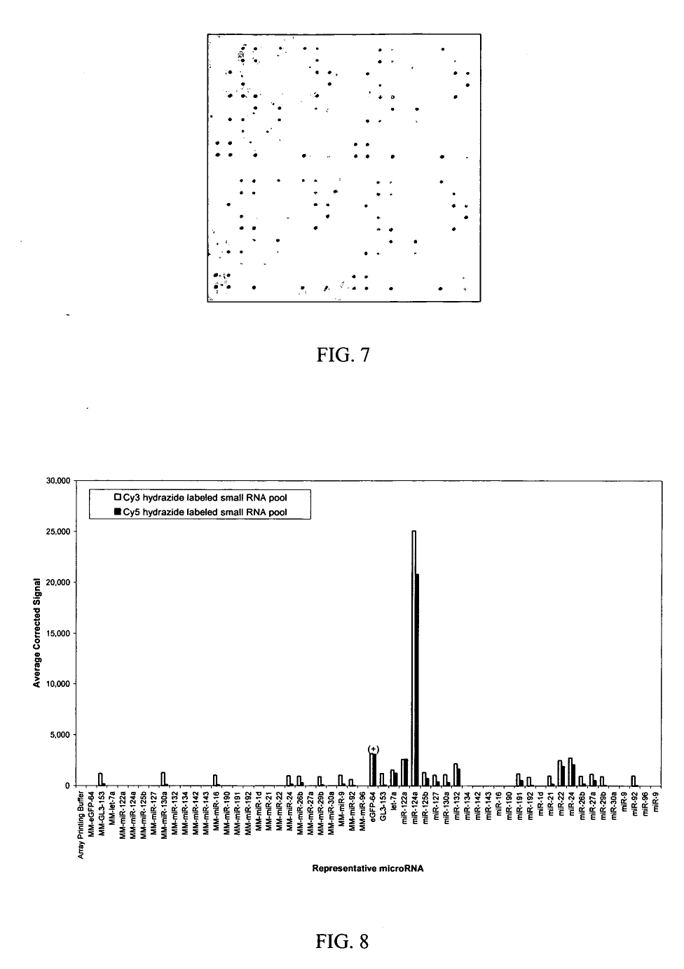

Total RNA was isolated from HeLa cells to serve as template for synthesizing cRNA according to standard procedures. A 2 μg sample of HeLa cRNA was fragmented and labeled with linear cationic CY3™ hydrazide as described in example 5. Following purification, the sample was hybridized to human oligo microarray slides (MWG Biotech Human Oligo test set) and analyzed for gene expression. The oligo set included sequences representing human housekeeping genes and negative controls. The microarray probed with the labeled RNA showed expression profiles consistent with expected results and demonstrate quality microarray data was attained using cationic hydrazide labeling reagents (FIG. 7).

### Example 12

### Microarray Hybridization of Cationic Hydrazide Labeled RNA

Cationic hydrazide labeling results in quality RNA hybridization data (for example, cRNA or microRNA). Using HeLa cells or mouse tissues, cRNA and “small RNA” (containing miRNA) were obtained using established methods (modified Eberwine antisense RNA amplification or MIRVANA™ miRNA isolation kit). RNAse III fragmented RNA, 5 μg cRNA or 1 μg “small RNA” was labeled according to procedures in Example 5 using biotin, CY™3, or CY™5 cationic hydrazides, and hybridized to appropriate printed arrays.

For hybridization, human oligo microarray slides (MWG Biotech Human Oligo test set) or microRNA oligo sets (designed from the mature microRNA sequences described in the Sanger institute microRNA Registry) were produced using established procedures. Oligo sets included a printing buffer control and known mismatch or plant sequence negative controls to represent non-specific hybridization.

The chart shown in FIG. 8 illustrates microRNA expression data obtained using microarray hybridization, with average corrected signal (Y axis) representing expressed levels of the specific printed sequence (X axis). This pool of “small RNA” from different tissues was also spiked with eGFP-64 synthetic RNA oligo (marked as (+) above bars) at a known level as a positive control for hybridization quality and sensitivity. Resulting expression of cationic hydrazide labeled RNAs corroborate reported profiles and demonstrate quality microarray data attained using the labeling methods outlined in Example 10.

Cationic hydrazide labeling and microarray hybridization of “small RNA” extracted from individual tissues (MIRVANA™ miRNA isolation kit) enable identification and measurements of relative abundance of tissue-specific miRNAs. For this type of profiling, microarrays are printed with a set of known miRNA capture sequences. The hybridization performance of labeled “small RNA” samples, derived from biologically relevant cells or tissues, reveals the specific miRNA profile for that particular specimen. Also, the competitive hybridization of two “small RNA” samples (for example, diseased versus normal or early vs. later developmental stages) labeled by spectrally distinct but compatible fluorophores (for example CY™3 and CY™5) may reveal specific miRNA profiles related to the diseased state. This knowledge base is necessary to determine the role of up or down regulation of microRNAs in relation to development or disease.

The labeling and hybridization of fragmented cRNA is a common technique in traditional microarray expression profiling analysis (for example, biotin labeling in the Affymetrix platform). The ability to efficiently end-label fragmented cRNA, using the cationic hydrazide technology, has the potential to greatly improve hybridization performance (compared to current enzymatic labeling methods that incorporate a variable number of labels within the cRNA) by minimizing the effect on hybridization Tₘ caused by the labeling method.

### Example 13

### Cationic Hydrazide Labeling of DNA

Fluorescent labeling of DNA, using aldehyde-specific (hydrazine) reagents, can be achieved by the introduction of aldehyde groups into the DNA by partial depurination (Proudnikov et al. 1996). Double stranded DNA (sheared salmon sperm DNA; 0.02 μg/μl) was treated with an alkylating agent (LABEL IT® biotin; 0.02 μg/μl according to recommended procedure, Mirus Bio, Madison, Wis.). CY™3 hydrazide was added to the modified DNA (final concentration 22 μM) and heated at 95° C. for 15 minutes to promote depurination of the DNA at the alkylated sites. The sample was incubated an additional 2 hours at room temperature to ensure completion of the hydrazide labeling reaction and purified by ethanol precipitation in the presence of glycogen. The purified DNA pellet was resuspended in buffer and spectrophotometrically assessed for CY™3 labeling efficiency. DNA samples undergoing this treatment resulted in significant label incorporation, with an average of 122.6 pmol CY™3/μg DNA. DNA, without the chemical introduction of abasic sites, does not react with hydrazide reagents and results in no detectable CY™3 signal.

### Example 14

### Synthesis of Vicinal-Heterosubstituted Amino-Endlabeling Reagent

Labeling reagents 30, 33, 34, 35 and 36 with reactive moieties I, II (general structure IV) were prepared from available protected derivatives of cysteine and 2,3-diaminopropionic acid, respectively.

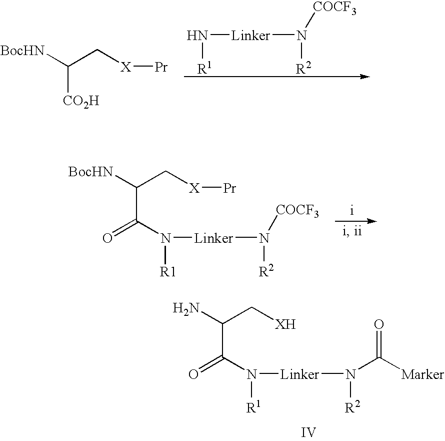

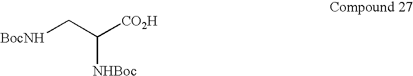

2,3-Diaminopropionic acid (Aldrich) (2.5 g, 17.79 mmol) was Boc-protected by treatment with Boc₂O (7.843 g, 35.94 mmol) in cold dioxane-H₂O 2:1 mixture (75 mL) in the presence of K₂CO₃ (12.294 g, 89 mmol). After addition of Boc₂O the cooling bath was removed and the reaction mixture was stirred for 10 h at RT. All volatiles were evaporated in vacuum. The product was suspended in H₂O, acidified to pH 1 with 10% KHSO₄, extracted with EtOAc, washed with brine and dried (MgSO₄). Yield 5.35 g, 99%. MS 305 (M+H⁺), 327 (M+Na⁺), 631 (2M+Na⁺).

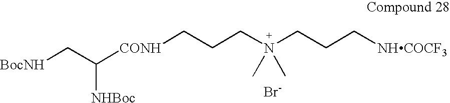

The amino-precursor 7 (388 mg, 0.890 mmol) was Boc-deprotected by stirring in TFA (2 mL) for 30 min, concentrated and dried in vacuum, and the residue was dissolved in DMF (1.5 mL). Compound 27 (270 mg, 0.89 mmol) was activated with HBTU (337 mg, 0.89 mmol) in presence of DIEA (345 mg, 2.67 mmol) in DMF (3 mL) for 5 min and added to 7. The reaction was stirred for 3 h at RT, DMF was removed under vacuum The residue was dissolved in a minimum of MeOH and the product was precipitated with Et₂O. MS: 542.4 (M⁺). The crude material was used directly in the next step.

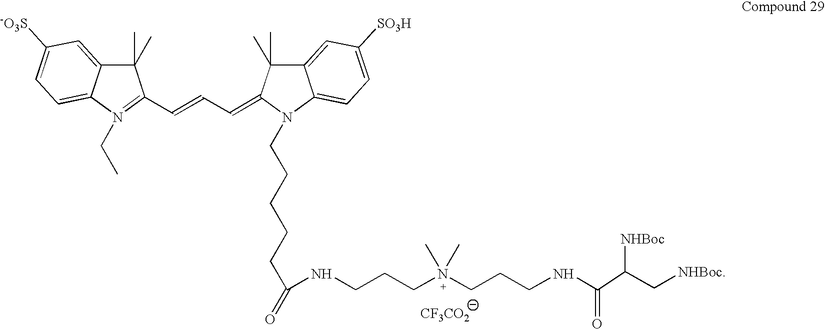

Compound 28 (554 mg, 0.89 mmol) was COCF₃ deprotected by stirring with K₂CO₃ (1.8 mmol, 248 mg) in MeOH—H₂O 3:2 mixture (3 mL) for 10 h at RT. The solvent was removed under vacuum. The product was dissolved in EtOH, filtered and concentrated in vacuum. MS: 446.4 (M⁺). A portion of the deprotected amine (7.2 mg, 0.0136 mmol) was stirred for 2 h with Cy3-NHS ester (10 mg, 0.0137 mmol) in DMF (200 μL) as described for compound 14. The product was precipitated with Et₂O and purified by HPLC using a C-18 Aquasil column (250×21.2 mm), mobile phase MeOH (TFA 0.1%)/H₂O (TFA 0.1%), flow 15 mL/min). MS: 1058 (M+H⁺), 1096 (M+Na⁺).

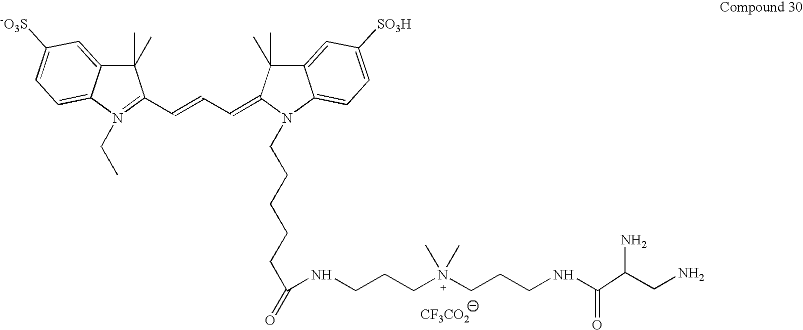

Compound 29 was treated with a mixture of TFA-CH₂Cl₂ 2:1 (3 mL) for 45 min and concentrated under vacuum. The residue was dissolved in H₂O (3 ml) and lyophilized. Yield 8 mg. MS: 858.6 (M+H⁺). MS: 858 (M+H⁺). Anal. HPLC (C-18 Aquasil column (250×4.6 mm), mobile phase MeOH (TFA 0.1%)/H₂O (TFA 0.1%), flow 1 mL/min) proved purity of 99%.

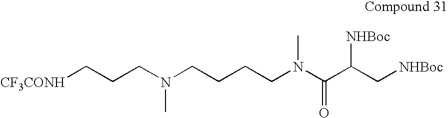

Compound 31 was prepared from compound 13 (19 mg, 0.053 mmol) following Boc deprotection by stirring with CH₂Cl₂-CF₃CO₂H mixture (1:1, 0.2 mL) for 0.5 h. The reaction mixture was dried under vacuum, dissolved in DMF (0.2 mL) and DIEA (21 mg, 0.16 mmol) was added. Di-Boc aminoacid 27 (16.1 mg, 0.053 mmol) was activated with a mixture of i-BuOCOCl (7.2 mg, 0.053 mmol) and DIEA (7.3 mg, 0.057 mmol) in DMF (0.2 mL) at 0° C. for 5 min, and added to the deprotected amine. The reaction mixture was stirred for 2 h at RT, diluted with Et₂O (10 mL), the precipitate was removed, and the supernatant was washed with 1N NaOH, brine, and dried (MgSO₄). MS: 556.2 (M+H⁺), 578.4 (M+Na⁺). The product was utilized in preparation of 32 without further purification.

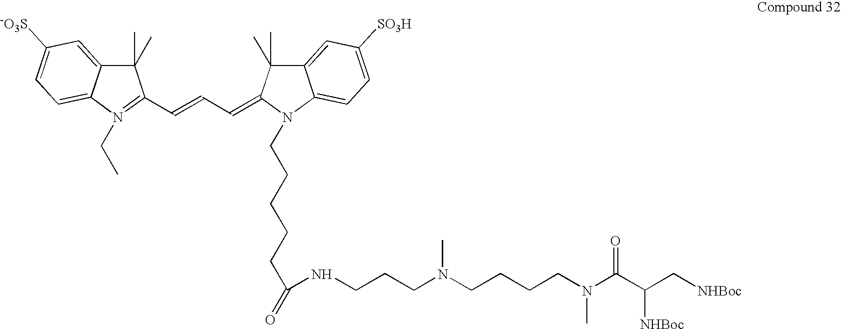

The crude compound 31 was COCF₃-deprotected by refluxing for 4 h in the mixture of CH₃OH (3 mL) and conc. aqueous NH40H (2 mL). The reaction mixture was concentrated in vacuum, dissolved in DMF (0.2 mL) and stirred with Cy3-NHS ester (15.6 mg, 0.0215 mmol) for 3 h. The product was precipitated with Et₂O (2 mL) and purified by HPLC using C-18 Aquasil colum (250×21.2 mm), mobile phase MeOH (TFA 0.1%)/H₂O (TFA 0.1%), flow 15 mL/min). MS: 1073 (M+H⁺).

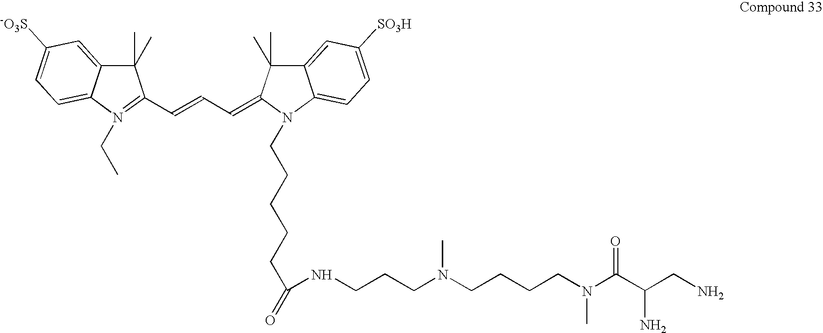

Compound 33 was obtained from 32 which was Boc-deprotected by stirring in a mixture of TFA (1.5 mL), CH₂Cl₂ (2 mL) for 45 min. The product was concentrated and dried in vacuum. Yield 6.2 mg. Anal. HPLC (C-18 Aquasil column (250×4.6 mm), mobile phase MeOH (TFA 0.1%)/H₂O (TFA 0.1%), flow 1 mL/min) gave purity 99%.

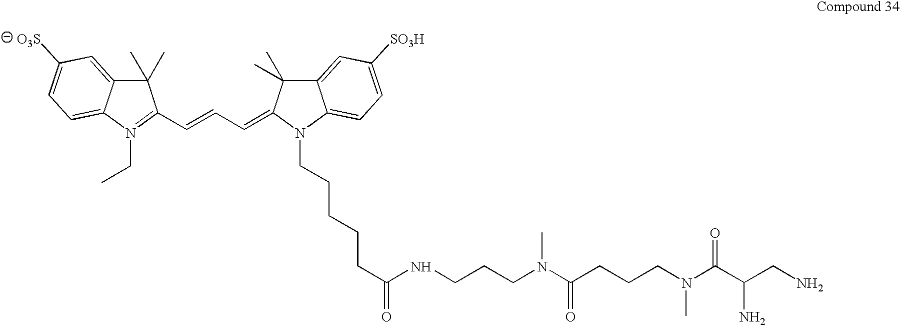

Similarly to compound 33, compound 34 was prepared from Cy3-NHS ester, amide linker H₂N(CH₂)₃N(CH₃)CO(CH₂)₃N(Boc)CH₃, and diBoc-protected amino acid 27. The product was purified by HPLC (C-18 Aquasil column (250×21.2 mm), mobile phase MeOH (TFA 0.1%)/H₂O (TFA 0.1%), flow 15 mL/min). MS: 886 (M+H⁺).

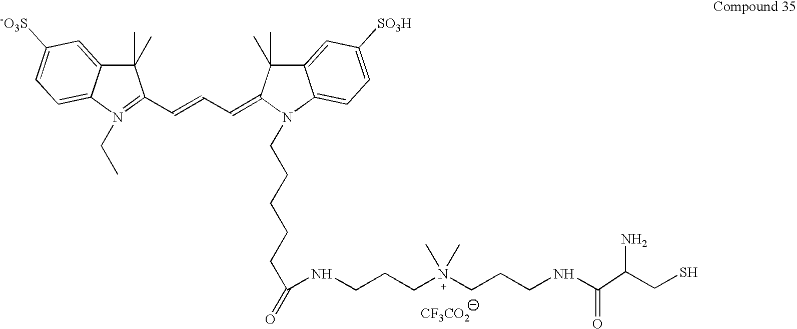

Similarly to compound 30, compound 35 was prepared from Cy3-NHS ester, precursor 28 and Boc-Cys(Trt)-OH aminoacid (NovaBiochem). The respective product of condensation (5 mg) was Trt and Boc-deprotected by 1 h stirring in a deprotection solution of TFA (1.840 mL), H₂O (0.06 mL), 1,2-ethanedithiol (0.06 mL), and triisopropylsilane (0.04 mL). The product was purified by HPLC (C-18 Aquasil column (250×21.2 mm), mobile phase MeOH (TFA 0.1%)/H₂O (TFA 0.1%), flow 15 mL/min). MS: 875.6 (M+H⁺).

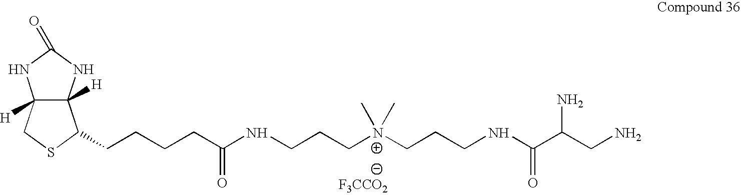

Similarly to compound 30, compound 36 was prepared from biotin-NHS ester and compound 28. Following COCF₃ deprotection with K₂CO₃ the crude product (12 mg, 0.023 mmol) was stirred with biotin-NHS ester (6.5 mg, 0.019 mmol) for 3 h. The product was precipitated with Et₂O (10 mL) and Boc-deprotected by stirring with a mixture of CH₂Cl₂-TFA (3:4, 0.7 mL) for 45 min. The product was concentrated in vacuum and purified by HPLC (C-18 Aquasil column (250×4.6 mm), mobile phase ACN (TFA 0.1%)/H₂O (TFA 0.1%), flow 15 mL/min). MS: 472.4 (M+).

### Example 15

### Labeling of siRNA Oligonucleotide with Reagents 30, 33, 34 and 36

Freshly prepared 0.1 M NaIO₄ solution (6 μL) was added to a solution of GL-3 siRNA (6 μg) in nuclease-free H₂O (30 μL). After the solution was incubated for 1 h at RT, the oxidized GL-3 was precipitated with 15 volumes of 2% solution of LiClO₄ in acetone for 20 min, centrifuged for 25 min at 14,000 rpm. Pellets were washed with acetone (2×200 μL) and dissolved in nuclease-free water (240 μL). The solution was split in 6 portions (6×40 μL) for 6 separate labeling experiments.

For each labeling experiment, the following reagents were added to the reaction tube:
a) KHPO₄ 0.1M buffer with pH=7.55, 1.5 μL;
b) Aqueous solutions of labeling reagents in concentration 1.29 mM, 0.62 μL (5 fold excess), or 1.24 μL (10 fold excess); and,
c) Nuclease-free water 33 μL.

The total volume of the reaction mixtures was approximately 75 μL. Reactions were carried on for 1 h at RT, or at 45° C.

The labeled GL-3 product was precipitated by the procedure described in Example 5. The reaction mixture was diluted 2× with an aqueous solution of glycogen that was used in amount of 1 μg of glycogen per 5 μL of the reaction mixture. Therefore, to each reaction mixture was added glycogen (75/5=15 μg or 3 μL of stock glycogen solution with concentration 5 μg/μL,) and H₂O (72 μL). The NaCl solution (5 M solution in amount equal to 0.1 of the obtained volume (75+72+3)×0.1=15 μL) was added followed by 2.5 volumes of ice cold EtOH ((150+15)ₓ2.5=412.5 μL).

The labeling density for compounds 30, 33, 34 and 36 was assessed by the ratio of absorption at A260/A550. The labeling density for compound 36 was assessed by gel electrophoresis by streptavidin gel shift of biotin-labeled RNA. The labeling density for Compounds 30, 33, and 36 was above 80% with 5 times excess and above 90% with 10 times excess of labeling reagent. Reagent 34 doesn't comprise basic nitrogen in a linker and is less protonated in aqueous media than 33, therefore it is much less reactive. It takes 5 times larger excess of 34 and 2 h to obtain the same labeling result as with 30 and 33.

### Example 16

### Labeling of Gl-3 RNA Oligonucleotide with Compound 35 at Basic pH

Freshly prepared 0.1 M NaIO₄ solution (4.5 μL) was added to a solution of GL-3 (9 μg) in nuclease-free H₂O (41.4 μL) and NaOAc buffer (0.6 μL, 1.5 M, pH=4.63). Solution was incubated for 1 h at room temperature. The oxidation was stopped by addition of Na₂SO₃ solution (4.5 μL, 0.15 M) without isolation of oxidized GL-3. Due to Na₂SO₃ presence in the reaction mixture at concentration 11.3 mM, the solution became strongly basic (pH=9). The resulting solution (60 μL) was divided in 3 parts for 3 separate experiments. The solution of oxidized G1-3 (20 μL) was diluted with nuclease-free water (174.9 μL) and treated with compound 36 (5.05 μL of 0.95 mM solution in H₂O) for 4 h. The labeled GL-3 pellets were precipitated and analyzed as described in Example 15, and showed a labeling efficiency of 22%.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

We claim:

1. A compound for labeling a polynucleotide comprising:
a) a detectable label;
b) an diamine group; and,
c) an affinity group.

2. The compound of claim 1 wherein the aldehyde reactive group is selected from the group consisting of: hydrazines, hydrazides, semicarbazides, and thiosemicarbazides, oxyamines, substituted diamines, and C-nucleophiles.

3. The compound of claim 1 wherein the affinity group is selected from the group consisting of: positively charged group, minor groove binder, major groove binder, intercalating group, nucleic acid binding protein, and nucleic acid binding peptide.

4. The compound of claim 1 wherein the detectable label comprises a molecule selected from the group consisting of fluorescence molecule, hapten, protein, peptide, biotin, and radioactive atom.

5. The compound of claim 4 wherein the fluorescent molecule is selected from the group consisting of: rhodamine, rhodamine derivative, fluorescein, fluorescein derivative, cyanine dye, cyanine dye derivative, hemi-cyanine dye, pyrene, lucifer yellow, BODIPY®, malachite green, coumarin, dansyl derivative, mansyl derivative, dabsyl derivative, NBD fluoride, stillbene, anthrocene, acridine, rosamine, TNS chloride, ATTO-TAG™, Lissamine™ derivative, ALEXA® dye, eosin, naphthalene derivative, ethidium bromide derivative, thiazole orange derivative, ethenoadenosine, Oregon Green®, Cascade Blue®, IR Dye, Thiazole Orange, BODIPY®-Fl, TAMRA, and green fluorescent protein.

6. The compound of claim 1 wherein the detectable label comprises a molecule selected from the group consisting of: reactive group, charged groups, alkyl groups, polyethyleneglycol, ligand, and peptide.

7. The compound of claim 1 further comprising a spacer.

8. The compound of claim 7 wherein the spacer is cationic.

9. A compound having the structure comprising:
D-B-A
wherein,
D comprises a detectable label selected from the group consisting of: fluorescence group, radioactive catom, hapten, immunogenic group, chemiluminescence-emitting compound, biotin, peptide, and protein;
B comprises an affinity group selected from the group consisting of: positively charged group, minor groove binder, major groove binder, intercalating group, nucleic acid binding protein, and nucleic acid binding peptide
A comprises an aldehyde reactive group is selected from the group consisting of: hydrazines, hydrazides, semicarbazides, and thiosemicarbazides, oxyamines, substituted diamines, and C-nucleophiles

10. A method for covalent attachment of a label to a polynucleotide comprising:
a) forming a labeling reagent comprising: a detectable label, an aldehyde reactive group, and an affinity group;
b) modifying the polynucleotide to contain an aldehyde; and
c) combining the labeling reagent with the modified polynucleotide.

11. The method of claim 10 wherein the aldehyde reactive group is selected from the group consisting of: hydrazines, hydrazides, semicarbazides, and thiosemicarbazides, oxyamines, substituted diamines, and C-nucleophiles.

12. The method of claim 10 wherein the affinity group is selected from the group consisting of: positively charged group, minor groove binder, major groove binder, intercalating group, nucleic acid binding protein, and nucleic acid binding peptide.

13. The method of claim 10 wherein the detectable label comprises a molecule selected from the group consisting of fluorescence molecule, hapten, protein, peptide, biotin, and radioactive atom.

14. The method of claim 13 wherein the fluorescent molecule is selected from the group consisting of: rhodamine, rhodamine derivative, fluorescein, fluorescein derivative, cyanine dye, cyanine dye derivative, hemi-cyanine dye, pyrene, lucifer yellow, BODIPY®, malachite green, coumarin, dansyl derivative, mansyl derivative, dabsyl derivative, NBD fluoride, stillbene, anthrocene, acridine, rosamine, TNS chloride, ATTO-TAG™, Lissamine™ derivative, ALEXA® dye, eosin, naphthalene derivative, ethidium bromide derivative, thiazole orange derivative, ethenoadenosine, Oregon Green®, Cascade Blue®, IR Dye, Thiazole Orange, BODIPY®-Fl, TAMRA, and green fluorescent protein.

15. The method of claim 10 wherein the detectable label comprises a molecule selected from the group consisting of: reactive group, charged groups, alkyl groups, polyethyleneglycol, ligand, and peptide.

16. The method of claim 10 wherein the labeling reagent further comprises a spacer.

17. The method of claim 16 wherein the spacer is cationic.
